(19) United States
(12) Patent Application Publication
(10) Pub. No.: US 2005/0257795 A1
(43) Pub. Date: Nov. 24, 2005

(54) Method and apparatus for locating superficial veins or specific structures with a LED light source

(76) Inventors: Yu Hsiu-Chen, Taipei (TW); Lin Chin-Liang, Taipei (TW)

Correspondence Address: BACON & THOMAS, PLLC, 625 SLATERS LANE, FOURTH FLOOR, ALEXANDRIA, VA, 22314, US

(21) Appl. No.: 11/125,266

(22) Filed: May 10, 2005

(30) Foreign Application Priority Data
May 20, 2004 (TW) 093114318
Jun. 1, 2004 (TW) 093208643

(51) Int. Cl. G01J 1/00; H01J 40/00; A61B 5/00; B65D 81/00

(52) U.S. Cl. 128/898; 250/200

(57) ABSTRACT

A light source locating and visualization apparatus comprises one or more than one LEDs capable to radiate a red light source with a wavelength of 600˜650 nm to locate superficial veins or specific structures; and a method of locating superficial veins with a LED light source comprises to proceed a skin-tightly radiating by using the source locating and visualization apparatus to emitting a red LED light source with wavelength of 600˜656 nm; to produce a lateral scattering and illumination effect by way of emitting red LED light source penetrating through a fat structure to cause reflection and scattering; and to locate superficial veins by naked eyes due to said superficial veins capable of absorbing red light spectrum easily and appearing as a located blood vessel shadow under the lateral scattering and illumination effect; therefore, physicians and nurse are capable by naked eyes to see the size, curve, and bifurcation of subcutaneous blood vessels visually and then select an appropriate injection position along the blood vessel for intravenous puncture easily.

### BACKGROUND OF THIS PRESENT INVENTION

1. Field of this Present Invention

This present invention related generally to a method and apparatus for locating superficial veins or specific structures with a LED light source, more particularly to a method and apparatus for by naked eyes visualizing blood vessel images with a red LED light source having a wavelength of 600˜650 nm.

2. Description of Prior Act

About one hundred years ago, physicians started using veinopuncture for blood extraction, drug delivery, blood transfusion, normal saline or other fluids. The prerequisite to do this is to locate blood vessels for intravenous insertion. Locating blood vessels is especially important during medical emergency in establishing fluid infusion line in the body of the patient.

Current clinical blood vessel locating methods and their shortcomings are described as follows:
1. Visual method to search and locate blood vessels visually.
   This method is suitable for patients having obvious superficial veins, but cannot generate images of the size, curve, and bifurcation of a blood vessel.
2. Touch method to search and locate blood vessels by touching the skin.
   This method needs rich experience of medical staff to identify the difference between blood vessel and fat structure, and is suitable for patients having thick subcutaneous fat. However, the success rate of this approach depends much on personal experience of the medical staff and, thus, is low.
3. Dissection position to search and locate blood vessels based on anatomy.
   This method depends on fixed relative position of blood vessels in some portion of the body, e.g. central venous puncture. However, difference between individual persons in anatomy exists and, thus, physicians are not capable to identify the position of a specific blood vessel accurately with this method. The success rate of this method is very low when veins are overlaid thick fat tissue.
4. Supersonic or doppler method to search and locate blood vessels supersonically.
   This method is used commonly by cardiovascular physicians. However, the apparatus is expensive and importable, and a monitor is required to display images. This method is not capable to provide a real-time and in situ mechanism for physicians to locate subcutaneous blood vessels.
   Another supersonic method is used clinically to locate blood vessels under a detector for vessel injection, but it cannot provide clear images of blood vessels status or bifurcation of subcutaneous blood vessels.
5. Infrared ray or near infrared ray to search and locate blood vessels by vessel temperature.
   Since subcutaneous blood vessels bring thermal energy through the body, the temperature of blood vessels is theoretically higher than surrounding tissues. An infrared ray or near infrared ray detector is used to visualize vessel images, which are displayed on a monitor for identification of blood vessel distribution. However, the apparatus is expensive and importable, and this method is not capable to provide a real-time and in situ mechanism for physicians to locate subcutaneous blood vessels for intravenous insertion.
6. Thermal sensor imaging materials or products e.g. stickers to search and locate blood vessels by detecting the temperature of blood vessels.
   When a thermal sensor imaging sticker is adhered on the skin above a specific blood vessel, it visualizes the blood vessel due to the irradiative nature of the heat from the blood vessel. However, the resolution and imaging quality of this method is poor and the detected position of the interested subcutaneous blood vessel is inaccurate. Further more, it takes a few minutes to conduct heat energy to the sticker to visualize the blood vessel and, thus, is not practical. The thermal sensor imaging sticker may not work for patients having very thick subcutaneous fat.
7. Photoacoustic method to identify subcutaneous blood vessels using infrared rays and shock wave with a specific bandwidth and frequency.
   This method is applied to patients with breast cancer or other blood vessel malfunctions. However, the apparatus of this method is very expensive and images can only be displayed on a monitor. This method is not capable to provide a real-time and in situ mechanism for physicians to locate subcutaneous blood vessels.
8. Fiber optic with halogen light source or called snake luminescent lamp to search and locate blood vessels of children's palms with a luminescent source.
   Due to full scale of light spectrum, this method has insufficient penetrative illumination in tissue and the apparatus is big, importable, expensive, and heat generating. The application is used only on palms of children via transmission mode.

### SUMMARY OF THIS PRESENT INVENTION

These prior methods and apparatus for visualization of blood vessels have their individual shortcomings and are not capable to assist physicians and nurses to view the size, curve, and bifurcation of subcutaneous blood vessels with naked eyes in a real-time manner when viewing the blood vessels, especially the vein.

Inaccurate intravenous insertion of needle often occurs when the blood vessel of a patient are not easily identified and located due to these overlying thick fat structure. In another case, needle insertion in inappropriate or curve blood vessels due to visual unidentifiable reasons may bring difficulty in fluid transfusion, or result in inserting the syringe needle to the vessel wall or piercing through the blood vessel so that the fluid flow out of the blood vessel into other structures.

The primary objective of this present invention is to locate superficial veins or other tissues of the body using a LED, xenon lamp bulb or tungsten lamp bulb as a light source, wherein a LED emitted with a visible red light wave is used as the light source for this purpose. The optical locating and visualizing apparatus of this present invention is characterized in its heavy light density, thin, light weights, short and small design and capable of displaying the size, curve, and bifurcation of subcutaneous blood vessels through the scattering and transmission and reflection mechanism of the red light in a real-time manner and assisting physicians and nurses to find the position of blood vessels visually that is appropriate for insertion.

Another objective of this present invention is to provide a method of using a red LED as the light source to locate superficial veins of the body. With the characteristics of hemoglobin in the vein to absorb red light spectrum and visually turn to be black color, red light to penetrate these overlying fat structure with its stronger penetrability and relative stronger contrast, a lateral scattering and illumination effect is produced during the light traveling, refraction and scattering of the red light when a red light source with a wavelength of 600˜650 nm is skin-tightly radiated through the fat structure. An obvious blood vessel shadow, especially the vein shadow, appears under the scattering illumination from the delivered red light, which facilitates the absorption of the red light by subcutaneous superficial blood vessels. With the method of this present invention, physicians and nurse are capable to see the size, curve, and bifurcation of subcutaneous blood vessels visually and then select an appropriate injection position along the blood vessel for intravenous puncture easily. This present invention provides a solution for physicians and nurses to locate superficial veins of patients who have thick fat structure.

The third objective of this present invention is to provide a method of using a LED light source to locate superficial veins of the body. The principle of “Persistent of Vision” occurs when a person sees an object is used in this present invention. Since different refraction angles are produced when superficial structures of the body is radiated with light sources of different wavelengths, LED light sources with at least two different wavelengths, such as red, green and blue light sources, that radiate alternatively based on an intermittent time lag produce different contours of a specific superficial structure under the skin due to the refraction angles formed by the light sources with different wavelengths. The visualization of contours caused by the intermittent time lag converts the static superficial structure image to an illusion of dynamic movement, by which the boundary of different superficial structures can be identified easily. The method and apparatus for locating superficial structures with a LED Light source is applicable to skin disease, superficial vein diseases, melanoma, and lipoma etc.

### BRIEF DESCRIPTION OF THE DRAWING FIGURES

FIG. 1 shows an appearance view of the light source locating and visualization apparatus of this present invention.

FIG. 2 shows a schematic view of the light source locating and visualization apparatus of this present invention that is attached with different accessories for use.

FIG. 3 shows a schematic view of the light source locating and visualization apparatus of this present invention that has a multi-color LED base and at least a LED chip.

FIG. 4 shows a schematic view of the method and its description of this present invention to use a red LED light source to locate superficial veins of the body.

FIG. 5 shows a state view concerning the use of the light source locating and visualization apparatus of this present invention and the embodiment of FIG. 4.

FIG. 6 shows a schematic view of another embodiment of the light source locating and visualization apparatus of this present invention.

FIG. 7 shows a state view concerning the use of the light source locating and visualization apparatus of this present invention and the embodiment of FIG. 6.

FIG. 8 shows a schematic view of another method and its description of this present invention to use a red LED light source to locate superficial veins of the body.

FIG. 9 shows a state view concerning the use of the light source locating and visualization apparatus of this present invention and the embodiment of FIG. 6.

FIG. 10 shows a schematic view of another embodiment of the light source locating and visualization apparatus as a ring-shaped apparatus of this present invention.

FIG. 11 shows a state view concerning the use of the ring-shaped apparatus shown in FIG. 10 of this present invention.

FIG. 12 shows a schematic view of another embodiment of the light source locating and visualization apparatus as a horseshoe-shaped apparatus of this present invention.

FIG. 13 shows a state view concerning the use of the horseshoe-shaped apparatus shown in FIG. 12 of this present invention.

FIG. 14 shows a schematic view of another embodiment of the light source locating and visualization apparatus as a compass-shaped apparatus of this present invention.

FIG. 15 shows a state view concerning the use of the compass-shaped apparatus shown in FIG. 14 of this present invention.

FIG. 16 shows a schematic view of the method and its description of this present invention to use a LED light source to locate specific superficial structures of the body.

### DETAILED DESCRITION OF THE PREFERRED EMBODIMENTS

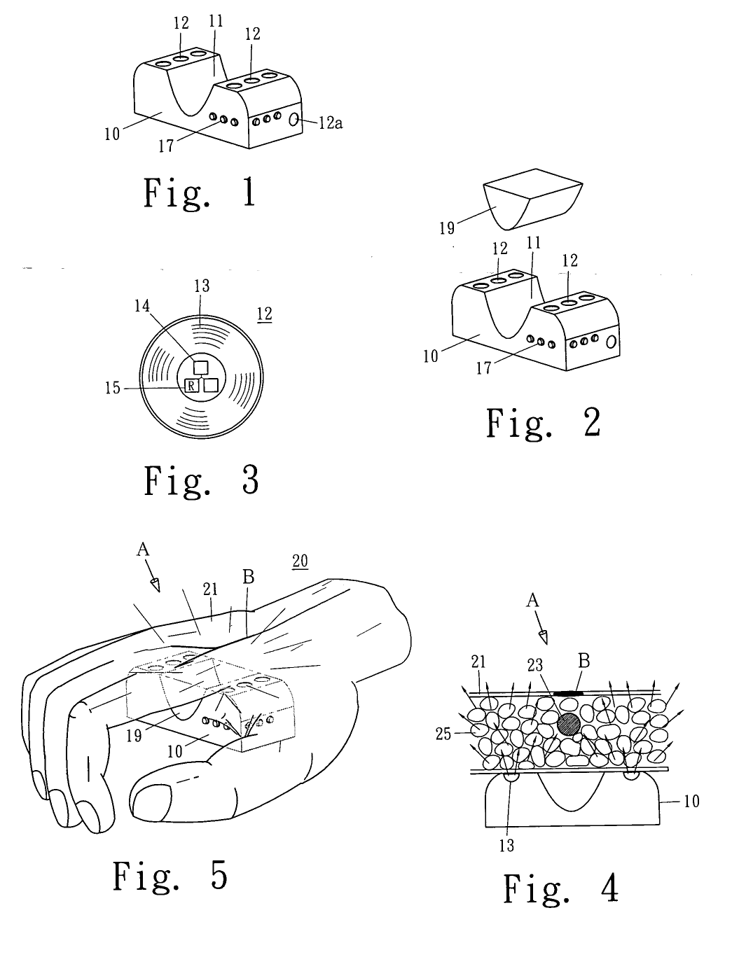

As shown in FIG. 1, a light source locating and visualization apparatus 10 of this present invention is an apparatus used to locate superficial veins or specific structures with a light source including LED, Xenon lamp bulb or tungsten lamp bulb, but preferably using a red LED as a light source. Said apparatus 10 is applicable to injection, intravenous injection, blood vessel searching and identification of the boundary between different superficial structures, and is capable to show the size, curve, and bifurcation of subcutaneous blood vessels in a real-time manner as shown in FIGS. 5 and 9, facilitating physicians and nurse by naked eyes to visually select appropriate position on a blood vessel for intravenous injection.

As shown in FIG. 1, an indented insertion groove 11, preferably shaped in indented U-sectional groove, V-sectional groove or the like, is disposed on the top of the light source locating and visualization apparatus 10 of this present invention.

One or more than one LED bases 12 are disposed on one side or each side of the insertion groove 11. As shown in FIG. 3, the LED base 12 has selectively a single red LED chip 15 to radiate red light with a wavelength of 600˜650 nm, or multiple LED chips 14 to radiate multiple lights with different wavelengths and colors, wherein at least a red LED chip 15 radiating red light with a wavelength of 600˜650 nm is required.

Each LED chip 14, including the red LED 15 and other chips, of each LED basis 12 must connect individual to each contact of an internal IC disposed in the light source locating and visualization apparatus 10. Thus, all LED base 12, part of LED base 12 or one LED base 12 is selectively controlled to radiate light by operating the control switch 17 of the light source locating and visualization apparatus 10. The LED base 12 is also selectively controlled to radiate a single red light or multiple lights, including white light and lights with other colors. With this mechanism, each LED base 12 is allowed in turn to radiate LED lights with different wavelengths and colors on a fixed interval.

As shown in FIGS. 2 and 5, an accessory 19 with a shape matching with the indented insertion groove 11 of the light source locating and visualization apparatus 10 shall be used to fill up the indentation of the insertion groove 11 under different conditions of use. Alternatively, a strap 18 can be attached to the light source locating and visualization apparatus 10 so that users can use the light source locating and visualization apparatus 10 conveniently.

As shown in FIGS. 3 and 4, each LED base 12 of the light source locating and visualization apparatus 10 has an arc backlight panel 13 to enhance the radiation of the light source. Alternatively, as shown in FIG. 1 an additional LED base 12a for radiation of white light for illumination can be disposed on a side of the body of the light source locating and visualization apparatus 10.

The light source locating and visualization apparatus 10 of this present invention to locate superficial veins or specific structures of a body using a LED light source is based on the principles, i.e., when a red light is radiated from the light source locating and visualization apparatus 10 of this present invention, the red light is absorbed by the hemoglobin in blood vessels, and red light has stronger penetrability and is capable to penetrate through the fat structure in the superficial portion of body; and when a red light source with a wavelength of 600˜650 nm is skin-tightly radiated through the fat structure, the red light penetrate through the fat structure, a lateral scattering and illumination effect is produced due to the light conduction, refraction and scattering of the red light; consequently, an obvious blood vessel shadow, due to hemoglobin in the vein to absorb red light spectrum and visually turn to be black color, appears under the scattering illumination of the red light, which facilitates the absorption of the red light by subcutaneous superficial blood vessels.

Accordingly, the method of locating superficial veins with a LED light source comprises to proceed a skin-tightly radiating by using the source locating and visualization apparatus 10 to emitting a red LED light source with wavelength of 600˜650 nm; to produce a lateral scattering and illumination effect by way of said emitted red LED light source penetrating through a fat structure to cause reflection and scattering; and to locate superficial veins by naked eyes due to said superficial veins capable of absorbing red light spectrum easily and appearing as a located blood vessel shadow under the lateral scattering and illumination effect.

The method of this prevent invention has two different applications to locate superficial veins with a LED light source. The first method of locating superficial veins with a LED light source is shown as in FIGS. 4 and 5, which makes use of the stronger penetrability of the red light to penetrate through the fat structure 25 of a body 20 and the characteristic of the hemoglobin in the blood vessel 23 to absorb the red light. Therefore, when the visual angle is in the direction A, a red light source radiating from the position opposite to the direction A can by naked eyes visualize the blood vessel shadow B on the skin 21 above the blood vessel 23 opposite to the light source.

As shown in FIG. 5, when a palm 20 of a patient contact tightly with the light source locating and visualization apparatus 10 and the apparatus 10 radiates a red light with a wavelength of 600˜650 nm, a located blood vessel shadow B appears on the skin 21 of the back side of the palm 20.

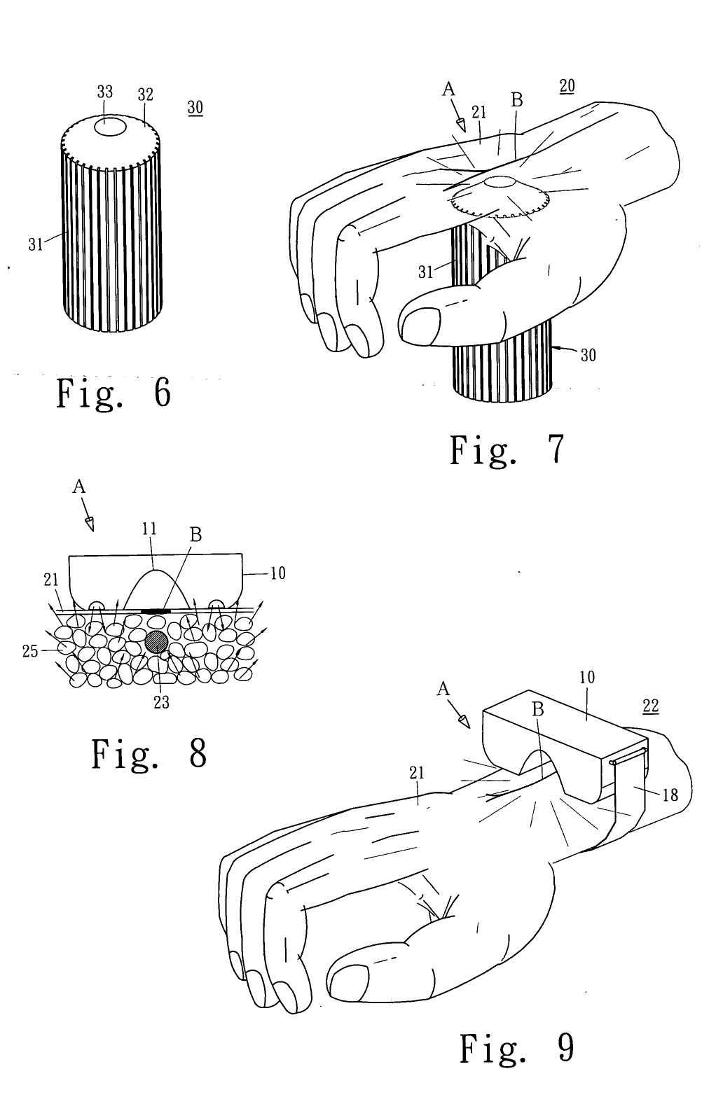

In application, another embodiment of the light source locating and visualization apparatus 30 of this present invention is shown as in FIG. 6, which has a column body 31 and one LED bases 33 is disposed on the top 32 of the column body 31.

As shown in FIG. 7, when a palm 20 of a patient directly put on the top 32 of the light source locating and visualization apparatus 30, the palm 20 of the patient shall contact tightly with the light source locating and visualization apparatus 30. After the apparatus 30 radiates a red light with a wavelength of 600˜650 nm, a located blood vessel shadow B shall obviously appear on the skin 21 of the back side of the palm 20.

So that physicians and nurse are capable by naked eyes to see the size, curve, and bifurcation of subcutaneous blood vessels in a real-time manner, and then select an appropriate injection position along the blood vessel for intravenous puncture easily. Particularly, this present invention provides a solution for physicians and nurses to locate superficial veins of patients who have thick fat structure.

The second method of locating superficial veins with a LED light source is shown as in FIGS. 8 and 9, which locates blood vessels 23 via scanning. When the fat structure 25 in the superficial portion of a body, e.g. a palm 20, is radiated with a linear light source, part of the light penetrates through the fat structure 25 while other lights produce a reflection and laterally scattering effect and, thus, produce a lateral illumination effect.

Due to many patients' superficial vein vessels 23 being covered by fat structure 25 hardly can be identified visually by naked eyes under white light. The light radiated from a red LED light source, however, is capable to penetrate through the fat structure 25 and produce reflection lateral scattering effect in the deeper layer of the fat structure 25. With this mechanism, the blood vessel 25 by the side of a single light source or in the middle of two light sources is presented in the form of a located blood vessel shadow B.

Therefore, when a strap 18 is attached to the light source locating and visualization apparatus 10 and used to contact tightly on the arm 22 of a patient, so that the light radiated from the light source locating and visualization apparatus 10 does not leak from the side. When the light source locating and visualization apparatus 10 radiates a red light with a wavelength of 600˜650 nm, an obvious located blood vessel shadow B in the direction of the visual angle A appears on the skin 21 of the portion of the arm 22 in the area of the insertion groove 11 of the light source locating and visualization apparatus 10.

With the second method of locating superficial veins with a LED light source mentioned above, due to an obvious position of blood vessels capably visualized by naked eyes in the area of the insertion groove 11, physicians and nurse are capable to identify the path, size, curve, and bifurcation of vessels 23 in the subcutaneous structure of a patient and definitely select appropriate position on a vessel for insertion.

Therefore, the light source locating and visualization apparatus 10 of this present invention can solve the problem of unidentifiable blood vessels of patients who have allows physicians and nurse to actually locate appropriate position of a vessel for insertion in a real-time and in-situ manner.

In application, another embodiment of the light source locating and visualization apparatus 40 of this present invention is shown as in FIG. 10, which is a ring-shaped apparatus provided with two LED bases 41 separated a distance to be formed as an insertion area 11a for injection, and a contacting ring 48 connected to the two separated LED bases 41.

As shown in FIG. 11, when the ring-shaped apparatus 40 is attached tightly on the arm 22 of a patient, an obvious located blood vessel shadow B shall obviously appear on the skin 21 of the portion of the arm 22 and in the insertion area 11a of the ring-shaped apparatus 40, after a red light with a wavelength of 600˜650 nm emitted from the two separated LED bases 41 of the ring-shaped apparatus 40,

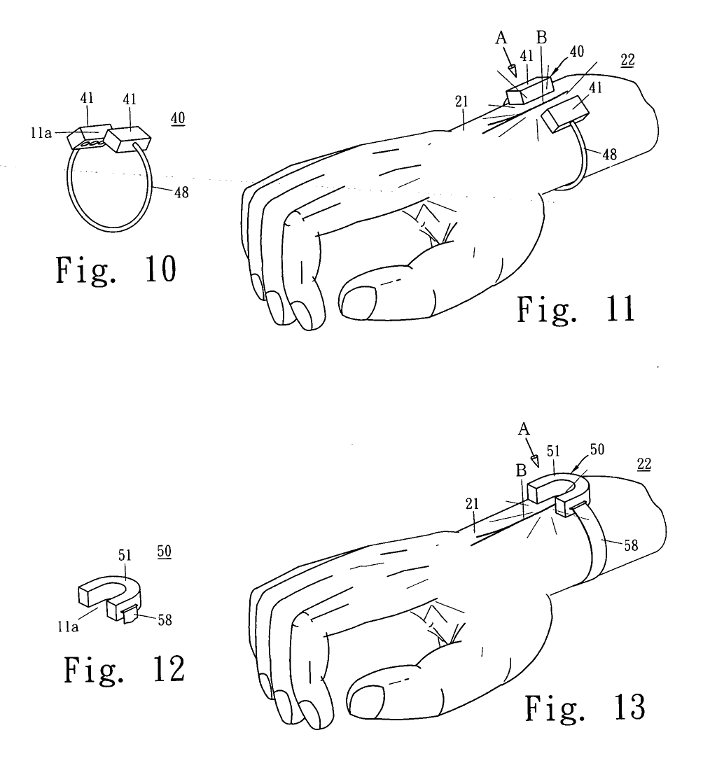

Further another embodiment of the light source locating and visualization apparatus 50 of this present invention is shown as in FIG. 12, which is a horseshoe-shaped apparatus provided with a horseshoe-shaped LED bases 51 with an insertion area 11a for injection, and a strap 58 connected to both sides of the horseshoe-shaped LED bases 51.

As shown in FIG. 13, when the horseshoe-shaped apparatus 50 is attached tightly on the arm 22 of a patient by its strap 58, an obvious located blood vessel shadow B shall obviously appear on the skin 21 of the portion of the arm 22 and in the insertion area 11a of the horseshoe-shaped apparatus 50, after a red light with a wavelength of 600˜650 nm emitted from the horseshoe-shaped LED bases 51 of the horseshoe-shaped apparatus 50.

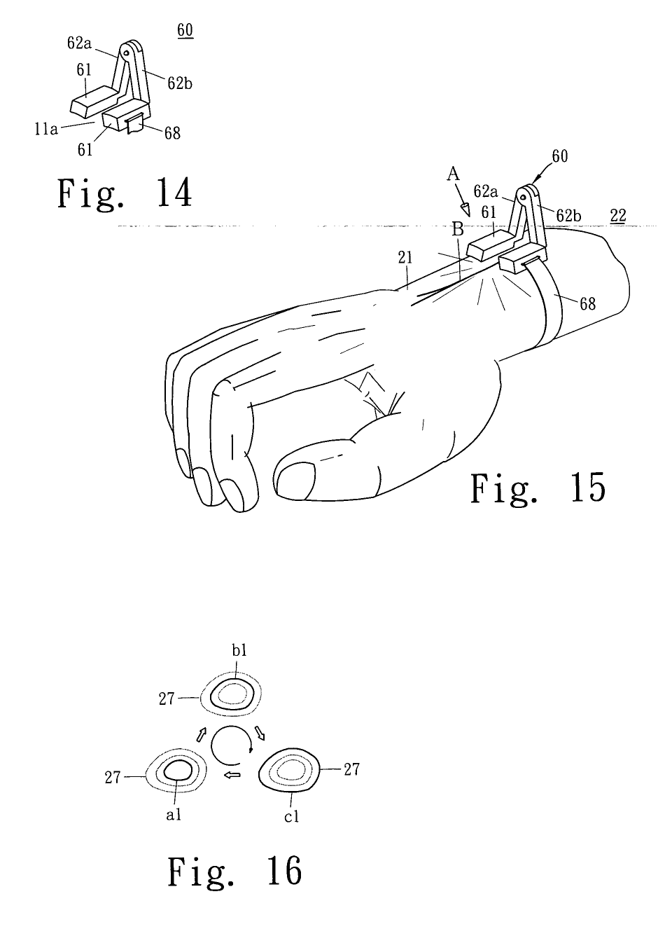

More embodiment of the light source locating and visualization apparatus 60 of this present invention is shown as in FIG. 14, which is a compass-shaped apparatus provided with two LED bases 61 respectively extended with a corresopnding connected rod 62a and 62b to be pivoted together and functioned as a compass to capably separate the two LED bases 61 with a distance as an insertion area 11a for injection, and a strap 68 connected to the two LED bases 61.

As shown in FIG. 15, when the compass-shaped apparatus 60 is attached tightly on the arm 22 of a patient by its strap 68, an obvious located blood vessel shadow B shall obviously appear on the skin 21 of the portion of the arm 22 and in the insertion area 11a of the compass-shaped apparatus 60, after a red light with a wavelength of 600˜650 nm emitted from the horseshoe-shaped LED bases 61 of the compass-shaped apparatus 60.

This present invention also makes use of the principle of Persistent of Vision that occurs when a person sees an object. Since different refraction angles are produced when superficial structures of the body is radiated with light sources of different wavelengths, LED light sources with at least two different wavelengths that radiate alternatively based on an intermittent time difference produce different contours of a specific superficial structure due to the refraction angles formed by the light sources with different wavelengths. The visualization of contours caused by the intermittent time difference converts the static superficial structure image to an illusion of dynamic movement of cells, by which the boundary of different superficial structures can be identified easily.

With this mechanism, the light source locating and visualization apparatus 10 is capable of identification or locating of superficial structure. As shown in FIG. 16, when the light source locating and visualization apparatus 10 alternatively radiates LED lights with different wavelengths and colors in an intermittent time difference manner, contours of the superficial structure 27 are shown in form of the first image boundary a1, second image boundary b1 and third image boundary c1 in circles.

The visualization of contours caused by the intermittent time difference converts the static superficial structure image to an illusion of dynamic movement of cells, by which the boundary of different superficial structures can be identified easily.

The method and apparatus for locating superficial veins or specific structures with a LED light source of this present invention has a wide range of applications, including the differentiation of benign fibromas and malignant tumors in a normal tissue. The benign fibroma and malignant tumor have different tissues. The content and distribution of blood capillaries in individual tissues are also different. The tissues of benign fibromas and malignant tumors are different from normal tissues. By radiating lights with different wavelengths from the light source locating and visualization apparatus 10, boundaries of normal tissues, benign fibromas and malignant tumors are distinguished clearly. The method for locating specific superficial structures of the body with a LED light source is also applicable to the identification of skin disease, melanoma, and lipoma.

1. A method for locating superficial veins with a LED light source, which comprising to proceed a skin-tightly radiating by emitting a red LED light source with wavelength of 600˜650 nm; to produce a lateral scattering and illumination effect by way of said emitted red LED light source penetrating through a fat structure to cause reflection and scattering; and to locate superficial veins by naked eyes due to said superficial veins capable of absorbing red light spectrum easily and appearing as a located blood vessel shadow under the lateral scattering and illumination effect.

2. The method for locating superficial veins with a LED light source as cited in claim 1, wherein the located blood vessel shadow of said superficial veins shall be appeared on a skin opposite to the skin where to proceed the skin-tightly radiating by emitting a red LED light source with wavelength of 600˜650 nm.

3. A method for locating specific structures with a LED light source, which comprising to proceed to radiate lights of different wavelengths to a specific structure in turn, based on an intermittent time lag to produce different contours of the specific structure due to refraction angles formed by the radiate light with different wavelengths; and to convert static superficial structure image to an illusion of dynamic movement due to the intermittent time lag, resulted in the boundary of different superficial structures shall be identified.

4. A light source locating and visualization apparatus applicable in locating superficial veins by naked eyes, which has an indented insertion groove and at least one or more than one LED light bases capable to radiate 600˜650 nm red LED light being disposed on the side of said insertion groove.

5. The light source locating and visualization apparatus as cited in claim 4, wherein at least one of LED light bases is capable to radiate LED lights with more than two colors.

6. The light source locating and visualization apparatus as cited in claim 4, wherein at least one of LED light bases is capable to radiate LED lights with more than two colors on an intermittent time difference basis.

7. The light source locating and visualization apparatus as cited in claim 4, wherein at least one xenon lamp bulb or tungsten lamp bulb is disposed on the side of said insertion groove.

8. The light source locating and visualization apparatus as cited in claim 4, wherein an accessory with a shape matching with said insertion groove is used to fill up an indentation of said insertion groove.

9. The light source locating and visualization apparatus as cited in claim 5, wherein an accessory with a shape matching with said insertion groove is used to fill up an indentation of said insertion groove.

10. The light source locating and visualization apparatus as cited in claim 6, wherein an accessory with a shape matching with said insertion groove is used to fill up an indentation of said insertion groove.

11. The light source locating and visualization apparatus as cited in claim 7, wherein an accessory with a shape matching with said insertion groove is used to fill up an indentation of said insertion groove.

12. The light source locating and visualization apparatus as cited in claim 4, wherein said light source locating and visualization apparatus further has a strap used to fix said apparatus on an arm of a patient.

13. The light source locating and visualization apparatus as cited in claim 4, wherein said light source locating and visualization apparatus has been made as a column body and one LED bases is disposed on its top of the column body.

14. The light source locating and visualization apparatus as cited in claim 4, wherein said light source locating and visualization apparatus is a ring-shaped apparatus provided with two LED bases separated a distance formed as an insertion area for injection and a contacting ring connected to the two separated LED bases.

15. The light source locating and visualization apparatus as cited in claim 4, wherein said light source locating and visualization apparatus is a horseshoe-shaped apparatus provided with a horseshoe-shaped LED bases with an insertion area for injection and a strap connected to both sides of the horseshoe-shaped LED bases.

16. The light source locating and visualization apparatus as cited in claim 4, wherein said light source locating and visualization apparatus is a compass-shaped apparatus provided with two LED bases respectively extended with a corresponding connected rod being pivoted together and functioned as a compass to capably separate the two LED bases with a distance as an insertion area for injection and a strap connected to the two LED bases.
